(19) United States
(12) Patent Application Publication
(10) Pub. No.: US 2025/0262399 A1
(43) Pub. Date: Aug. 21, 2025

(54) Cannula Device

(71) Applicant: Danise Lucas, Rio Rancho, NM (US)

(72) Inventor: Danise Lucas, Rio Rancho, NM (US)

(21) Appl. No.: 19/019,885

(22) Filed: Jan. 14, 2025

Related U.S. Application Data
(60) Provisional application No. 63/554,209, filed on Feb. 16, 2024.

(51) Int. Cl. A61M 16/06

(52) U.S. Cl. CPC A61M 16/0672

(57) ABSTRACT

A cannula device is disclosed, which is a modified nasal cannula comprising a facial portion of the nasal cannula, which is extra soft, 2.10 mt medical grade PVC tubing. Extra soft curved nasal prongs are installed on the terminal end of the soft tubing with a standard 4 mm internal diameter connector. This runs from the nose to just below the ears, and above the jawline. Another connector at this point allows the tubing to transition into a non-kink PVC tubing under the chin. A slide piece allows the cannula to be fitted to the user and secure the tubing, while allowing for adjustment. Below the slide piece, the non-kink tubing will then join into one piece with a standard flared connection to attach to the oxygen source. The device delivers medical oxygen at a flow rate of 1-6 liters/min and delivers FiO2 of 0.28-0.36, respectively.

### CROSS-REFERENCE TO RELATED APPLICATION

The present application claims priority to, and the benefit of, U.S. Provisional Application No. 63/554,209, which was filed on Feb. 16, 2024, and is incorporated herein by reference in its entirety.

### FIELD OF THE INVENTION

The present invention relates generally to the field of cannula devices. More specifically, the present invention relates to a nasal cannula featuring a combination of soft and non-kink tubing that fits comfortably around the cheeks and ears. Accordingly, this disclosure makes specific reference thereto the present invention. Nonetheless, it is to be appreciated that aspects of the present invention are also equally applicable to other like applications, devices and methods of manufacture.

### BACKGROUND

By way of background, this invention relates to improvements in cannula devices. Generally, people who wear nasal cannulas to have oxygen delivered to their respiratory system may have trouble dealing with the tubing. Non-kink tubing is stiff and harsh on the skin, often causing irritation on the cheeks and top of the ears. Thus, people may need to apply padding or fabric cushions to prevent skin breakdown. Further, if soft tubing is used, the cannula twists up under the chin and restricts airflow.

Oxygen therapy is usually delivered as a gas via an oxygen source. The oxygen is breathed through a nasal cannula or mask that covers the mouth and nose. The nasal cannula delivers oxygen through two small prongs that rest in the user's nostrils and leads to clear plastic tubing that hooks over the user's ears to be held in place and meets below the user's chin. The gas travels up through the nose and then down into the back of the throat where it is inhaled by the patient. Just normal breathing brings the extra oxygen into the lungs. The tubing is hooked up to one of several types of oxygen gas supply sources. However, one disadvantage associated with the tube is that flexible, normal movement of the user can result in the tube being blocked or restricted owing to kinking, bending, or twisting. A kink in the oxygen tube will restrict the oxygen flow and possibly be harmful to the user.

Accordingly, there is a demand for an improved cannula device that features soft, non-kink tubing for users. More particularly, there is a demand for a cannula device that enables users to maintain comfort without restricting airflow.

Therefore, there exists a long felt need in the art for a cannula device that provides users with a nasal cannula featuring soft tubing that fits comfortably around the checks and cars. There is also a long felt need in the art for a cannula device that transitions into the non-kink tubing below the cars, featuring a slide adjustment under the chin to accommodate individual fit. Further, there is a long felt need in the art for a cannula device that offers tubing that is not too soft so that it does not twist, but not too hard so that it does not hurt the wearer's face or cause skin irritation. Moreover, there is a long felt need in the art for a device that enables users to maintain comfort without restricting airflow from the oxygen system due to patient movement. Further, there is a long felt need in the art for a cannula device that also includes extra soft nasal prongs installed on a terminal end. Finally, there is a long felt need in the art for a cannula device that delivers medical oxygen at a flow rate of up to 6 liters per minute of supplemental oxygen.

The subject matter disclosed and claimed herein, in one embodiment thereof, comprises a cannula device. The device is a modified nasal cannula that combines soft tubing around the face and cars, then transitions into non-kink tubing to deliver reliable oxygen flow. The cannula device comprises a facial portion of the nasal cannula, which is extra soft, 2.10 mt medical grade PVC tubing. Extra soft curved nasal prongs are installed on the terminal end of the soft tubing with a standard 4 mm internal diameter connector. This runs from the nose to just below the cars, and above the jawline. Another connector at this point allows the tubing to transition into a non-kink PVC tubing under the chin. A slide piece allows the cannula to be fitted to the user and secure the tubing, while allowing for adjustment. Below the slide piece, the non-kink tubing will then join into one piece with a standard flared connection to attach to the oxygen source. The device delivers medical oxygen at a flow rate of 1-6 liters/min and delivers FiO2 of 0.28-0.36, respectively.

In this manner, the cannula device of the present invention accomplishes all of the forgoing objectives and provides users with soft tubing on the skin and reliable non-kink tubing extending to the source. This device helps prevent skin irritation and breakdown. The device maintains comfort without restricting airflow.

### SUMMARY OF THE INVENTION

The following presents a simplified summary, in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a cannula device. The cannula device comprises a soft portion of the nasal cannula with nasal prongs for delivering oxygen. This portion runs from the nose to just below the ears, and above the jawline. The soft tubing transitions from this point into non-kink tubing that incorporates an adjustable slide piece under the chin for fitting it to the user. The tubing is then attached to an oxygen source.

In one embodiment, the cannula device is a modified nasal cannula that is soft on the face and ears of the user but non-kink to enable movement by the user and maintain air flow. Specifically, the cannula device is soft and flexible and provides proper comfort to the face areas of a user safely with no compromise in air flow.

In one embodiment, the cannula device is a small, flexible and hollow tube that contains two nasal prongs configured to sit just inside a person's nostrils. The tube is approximately 14-22 gauge. The tubing attaches to an oxygen source and delivers a steady stream of medical-grade oxygen to the user's nose. The cannula device is lightweight, easy to use, and affordable. Further, the cannula device is relatively efficient and simple in terms of design and implementation. Generally, the cannula device and the oxygen source that it connects to may be used in a variety of hospital settings, at home, or activities in public. Further, nasal cannulas are medical devices used when people are unable to get sufficient oxygen to keep their body functioning optimally, whether that is due to a condition like COPD, another respiratory disorder, or an environmental change.

In one embodiment, the cannula device is utilized with a portable oxygen system. The portable oxygen system is configured to generate a supply of oxygen or oxygen-enriched gas to a respiratory circuit for delivery to an airway of a user who wants portability. For example, in some embodiments, the portable oxygen system may be a portable oxygen tank. In other embodiments, the portable oxygen system may be an oxygen concentrator. Although the present invention is described in a medical setting being used by a patient, one of ordinary skill in the art would understand that the invention may be used for various applications where additional oxygen is needed or desired to help with breathing, for example, for use by a fireman, pilot, paramedic, athlete, or medical responder.

In one embodiment, the nasal cannula device comprises two parallel and hollow tubing portions which extend upwardly around the cars of a user then terminate at spaced nasal prongs, which are adapted to be inserted into the nostrils of a patient/user. Typically, the two nasal prongs sit below the user's nose. The two nasal prongs deliver oxygen directly into a user's nostrils. The opposing ends of the parallel tubing portions are positioned underneath a user's chin, where they merge together and form into a single tubing which is secured to an oxygen source.

In one embodiment, the two parallel and hollow tubing portions comprise a soft tubing portion and a hard tubing portion connected together for comfort and use. Specifically, the soft tubing portion encompasses the facial portion of the nasal cannula. The facial portion is the tubing portion that runs from the nose to around a user's ears and above the jawline. Typically, the soft tubing portion is made of an extra soft, 2.10 mt medical grade PVC tubing to provide a soft, flexible tubing which is lightweight and prevents irritation of a user's skin during use.

In one embodiment, the soft tubing portion comprises extra soft curved nasal prongs. The extra soft curved nasal prongs are installed on the terminal end of the soft tubing portion. The nasal prongs are molded from a very soft, pliable rubber, plastic or similar material to prevent irritation to the nasal passages of the patient/user. Typically, the nasal prongs are installed with a standard 4 mm internal diameter connector. The nasal prongs provide comfort for the user, when using the cannula device. The two nasal prongs are configured to sit just inside a person's nostrils. During use, two nasal prongs deliver oxygen directly into a user's nostrils.

In one embodiment, the soft tubing portion is then secured to a hard tubing portion. The hard tubing portion is secured to the soft tubing portion via a connector. Any suitable tube connector can be used as is known in the art. The connector allows the soft tubing portion to transition into the hard tubing portion.

In one embodiment, the hard tubing portion is a non-kink PVC tubing, that secures under a user's chin. The hard tubing portion prevents kinks and twisting but is lightweight during use. Typically, the hard tubing portion is fabricated from a material more rigid than the soft tubing portion material is made from. Generally, the hard tubing portion is made from a plastic, polymer, PVC material, or other suitable more rigid material than the soft tubing portion.

In one embodiment, a slide piece is attached to the hard tubing portion. The slide piece allows the hard tubing portion to be fitted to the user and secures the tubing, while allowing for adjustment. Typically, the slide piece allows the hard, non-kink tubing portion to be secured under a user's chin during use.

In one embodiment, below the slide piece, the two hard non-kink tubing portions will then join into one piece with a standard flared connection. Once connected through the standard flared connection, the hard tubing portion is then attached to the oxygen source.

In one embodiment, the cannula device delivers medical oxygen at a flow rate of 1-6 liters/min and delivers FiO2 of 0.28-0.36, respectively. Standard flow tubing delivers concentrations between 24% and 44% depending on oxygen flow rate (up to 6 liters). A high flow cannula delivers up to 15 L/min. and greater concentrations.

In one embodiment, the two tubing portions of the cannula device can be made of a wide array of materials, including, but not limited to, silicon, rubber, plastic or other polymer-based materials, or other suitable materials as is known in the art. The two tubing portions can be made of any suitable materials, as long as the hard tubing portion of the cannula device is very lightweight, resistant to kinking, and doesn't become twisted easily. Further, the soft tubing portion of the cannula device must be very flexible and soft to ensure the greatest performance and comfort for the patient/user. Accordingly, the cannula device prevents bending or twisting of the hard tubing portion, however, the soft tubing portion remains flexible and comfortable to prevent irritation during use. This is an improvement in comparison to conventional devices wherein the tubing can be twisted, bent, or become kinked, or causes irritation and is uncomfortable to wear.

In yet another embodiment, the cannula device comprises a plurality of indicia.

In yet another embodiment, a method of maintaining comfort without restricting airflow through a cannula is disclosed. The method includes the steps of providing a cannula device comprising a soft tubing portion and a hard tubing portion. The method also comprises securing extra soft curved nasal prongs on a terminal end of the soft tubing portion. Further, the method comprises positioning the nasal prongs within a user's nostrils. The method also comprises positioning the soft tubing portion around a user's ears. The method also comprises transitioning the soft tubing portion to the hard tubing portion. The method comprises utilizing a slide piece to secure the hard tubing portion underneath a user's chin. Finally, the method comprises attaching the hard tubing portion to an oxygen source for use.

Numerous benefits and advantages of this invention will become apparent to those skilled in the art to which it pertains, upon reading and understanding the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one embodiment of the cannula device of the present invention in use in accordance with the disclosed architecture;

FIG. 2 illustrates a perspective view of one embodiment of the cannula device of the present invention showing the device and components in accordance with the disclosed architecture;

FIG. 3 illustrates a perspective view of one embodiment of the cannula device of the present invention showing the softer tubing portion of the device in accordance with the disclosed architecture;

FIG. 4 illustrates a perspective view of one embodiment of the cannula device of the present invention showing the harder, non-kink tubing portion of the device in accordance with the disclosed architecture;

FIG. 5 illustrates a perspective view of one embodiment of the cannula device of the present invention showing the slide piece to fit the device to a user in accordance with the disclosed architecture; and

FIG. 6 illustrates a flowchart showing the method of maintaining comfort without restricting airflow through a cannula in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long felt need in the art for a cannula device that provides users with a nasal cannula featuring soft, non-kink tubing that fits comfortably around the checks and cars. There is also a long felt need in the art for a cannula device that transitions into the non-kink tubing below the cars, featuring a slide adjustment under the chin to accommodate individual fit. Further, there is a long felt need in the art for a cannula device that offers tubing that is not too soft so that it does not twist, but not too hard so that it does not hurt the wearer's face or cause skin irritation. Moreover, there is a long felt need in the art for a device that enables users to maintain comfort without restricting airflow from the oxygen system due to patient movement. Further, there is a long felt need in the art for a cannula device that also includes extra soft nasal prongs installed on a terminal end. Finally, there is a long felt need in the art for a cannula device that delivers medical oxygen at a flow rate of 2-4 liters/min.

The present invention, in one exemplary embodiment, is a novel cannula device. The cannula device comprises a facial portion of the nasal cannula, which is extra soft, 2.10 mt medical grade PVC tubing. Extra soft curved nasal prongs are installed on the terminal end of the soft tubing with a standard 4 mm internal diameter connector. This runs from the nose to just below the cars, and above the jawline. Another connector at this point allows the tubing to transition into a non-kink PVC tubing under the chin. A slide piece allows the cannula to be fitted to the user and secure the tubing, while allowing for adjustment. Below the slide piece, the non-kink tubing will then join into one piece with a standard flared connection to attach to the oxygen source. The device delivers medical oxygen at a flow rate of 1-6 liters/min and delivers FiO2 of 0.28-0.36,respectively. The present invention also includes a novel method of maintaining comfort without restricting airflow through a cannula. The method includes the steps of providing a cannula device comprising a soft tubing portion and a hard tubing portion. The method also comprises securing extra soft curved nasal prongs on a terminal end of the soft tubing portion. Further, the method comprises positioning the nasal prongs within a user's nostrils. The method also comprises positioning the soft tubing portion around a user's ears. The method also comprises transitioning the soft tubing portion to the hard tubing portion. The method comprises utilizing a slide piece to secure the hard tubing portion underneath a user's chin. Finally, the method comprises attaching the hard tubing portion to an oxygen source for use.

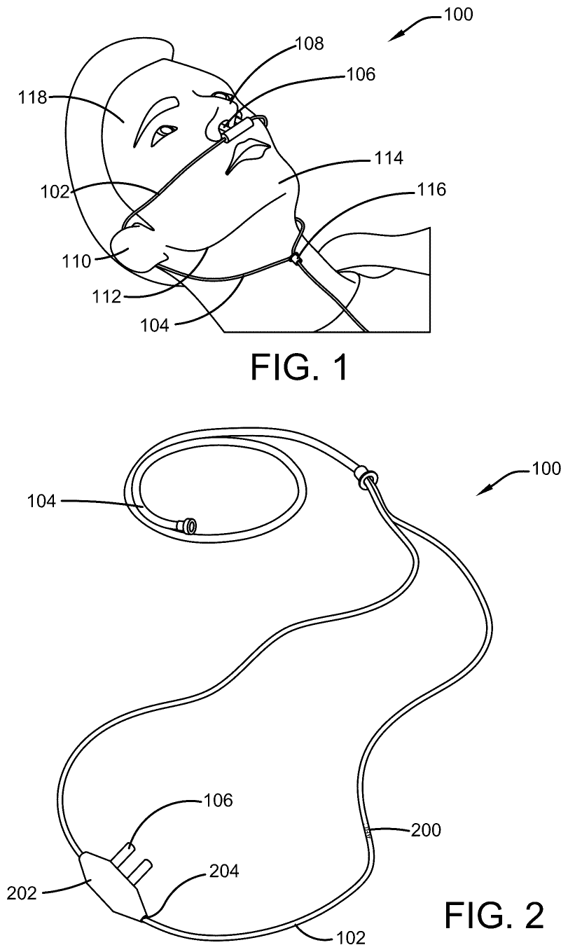

Referring initially to the drawings, FIG. 1 illustrates a perspective view of one embodiment of the cannula device 100 of the present invention. In the present embodiment, the cannula device 100 is an improved cannula device 100 that provides a user 118 with a modified nasal cannula. Specifically, the cannula device 100 comprises a soft tubing portion 102 and a hard tubing portion 104 connected together for comfort. The soft tubing portion 102 includes nasal prongs 106 for delivering oxygen. This portion 102 runs from the nose 108 to just below the cars 110, and above the jawline 112. A non-kink tubing portion 104 runs under the user's chin 114. A slide piece 116 allows the tubing 104 to be fitted to the user 118 and secure the tubing 104, while allowing for adjustment. The tubing 104 is then attached to an oxygen source 120.

Generally, the cannula device 100 is a modified nasal cannula that is not too soft so that it does not twist, yet not too hard so that it does not hurt the face of the user 118, while still allowing them to breathe. Thus, the device 100 enables users 118 to maintain comfort without restricting airflow from the oxygen system 120 due to patient movement. Specifically, the cannula device 100 is soft and flexible and provides proper comfort to the face areas of a user 118.

Further, the cannula device 100 is a small, flexible and hollow tube that contains two nasal prongs 106 configured to sit just inside a person's nostrils 108. The tube is approximately 14-22 gauge. The tubing 100 attaches to an oxygen source 120 and delivers a steady stream of medical-grade oxygen to the user's nose 108. The cannula device 100 is lightweight, easy to use, and affordable. Further, the cannula device 100 is relatively efficient and simple in terms of design and implementation. Generally, the cannula device 100 and the oxygen source 120 that it connects to may be used in a variety of hospital settings, at home, or activities in public. Further, nasal cannulas are medical devices used when people are unable to get sufficient oxygen to keep their body functioning optimally, whether that is due to a condition like COPD, another respiratory disorder, or an environmental change.

Additionally, the cannula device 100 is utilized with a portable oxygen system 120. The portable oxygen system 120 is configured to generate a supply of oxygen or oxygen-enriched gas to a respiratory circuit for delivery to an airway of a user 118 who wants portability. For example, in some embodiments, the portable oxygen system 120 may be a portable oxygen tank. In other embodiments, the portable oxygen system 120 may be an oxygen concentrator. Although the present invention is described in a medical setting being used by a patient, one of ordinary skill in the art would understand that the invention may be used for various applications where additional oxygen is needed or desired to help with breathing, for example, for use by a fireman, pilot, paramedic, athlete, or medical responder.

As shown in FIG. 2, the nasal cannula device 100 comprises two parallel and hollow tubing portions 102, 104 which extend upwardly around the cars 110 of a user 118 then terminate at spaced nasal prongs 106, which are adapted to be inserted into the nostrils 108 of a patient/user 118. Typically, the two nasal prongs 106 sit below the user's nose 108. The two nasal prongs 106 deliver oxygen directly into a user's nostrils 108. The opposing ends of the parallel tubing 104 portions are positioned underneath a user's chin 114, where they merge together and form into a single tubing 104 which is secured to an oxygen source 120.

In yet another embodiment, the cannula device 100 comprises a plurality of indicia 200. The soft tubing portion 102 of the device 100 may include advertising, a trademark, or other letters, designs, or characters, printed, painted, stamped, or integrated into the soft tubing component 102, or any other indicia 200 as is known in the art. Specifically, any suitable indicia 200 as is known in the art can be included, such as but not limited to, patterns, logos, emblems, images, symbols, designs, letters, words, characters, animals, advertisements, brands, etc., that may or may not be cannula, tubing, or brand related.

Further, the two parallel and hollow tubing portions comprise a soft tubing portion 102 and a hard tubing portion 104 connected together for comfort and use. Specifically, the soft tubing portion 102 encompasses the facial portion of the nasal cannula device 100. The facial portion is the tubing portion 102 that runs from the nose 108 to around a user's ears 110 and above the jawline 112. Typically, the soft tubing portion 102 is made of an extra soft, 2.10 mt medical grade PVC tubing to provide a soft, flexible tubing which is lightweight and prevents irritation of a user's skin during use.

Additionally, the soft tubing portion 102 comprises extra soft curved nasal prongs 106. The extra soft curved nasal prongs 106 are installed on the terminal end 204 of the soft tubing portion 102. The nasal prongs 106 are molded from a very soft PVC (pliable rubber, plastic or similar material) to prevent irritation to the nasal passages 108 of the patient/user 118. Typically, the nasal prongs 106 are installed with a standard 4 mm internal diameter connector 202. The nasal prongs 106 provide comfort for the user 118, when using the cannula device 100. The two nasal prongs 106 are configured to sit just inside a person's nostrils 108. During use, two nasal prongs 106 deliver oxygen directly into a user's nostrils 108.

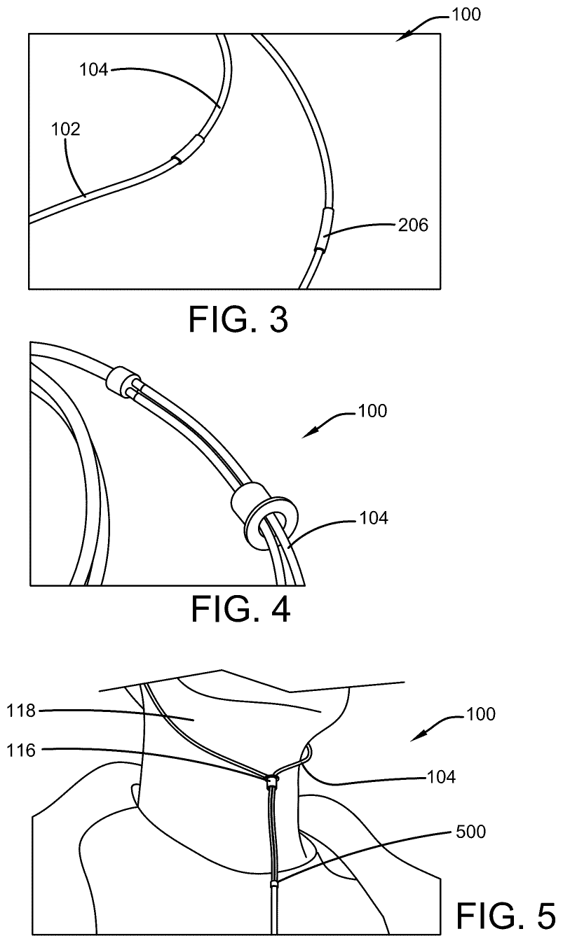

As shown in FIG. 3, the soft tubing portion 102 is then secured to a hard tubing portion 104. The hard tubing portion 104 is secured to the soft tubing portion 104 via a connector 206. Any suitable tube connector 206 can be used as is known in the art. The connector 206 allows the soft tubing portion 102 to transition into the hard tubing portion 104.

As shown in FIG. 4, the hard tubing portion 104 is a non-kink PVC tubing, that secures under a user's chin 114. The hard tubing portion 104 prevents kinks and twisting but is lightweight during use. Typically, the hard tubing portion 104 is fabricated from a material more rigid than the soft tubing portion 102 material is made from. Generally, the hard tubing portion 104 is made from a plastic, polymer, PVC material, or other suitable more rigid material than the soft tubing portion 102.

As shown in FIG. 5, a slide piece 116 is attached to the hard tubing portion 104. The slide piece 116 allows the hard tubing portion 104 to be fitted to the user 118 and secures the tubing 104, while allowing for adjustment. Typically, the slide piece 116 allows the hard tubing portion 104 to be secured under a user's chin 114 during use.

Further, below the slide piece 116, the two hard tubing portions 104 will then join into one piece with a standard flared connection 500. Once connected together through the standard flared connection 500, the hard tubing portion 104 is then attached to the oxygen source 120.

Additionally, the cannula device 100 delivers medical oxygen at a flow rate of 2-4 liters/min and delivers FiO2 of 0.28-0.36, respectively. Standard flow tubing delivers concentrations between 24% and 44% depending on oxygen flow rate (up to 6 liters). A high flow cannula delivers up to 15 L/min. and greater concentrations.

Furthermore, the two tubing portions 102, 104 of the cannula device 100 can be made of a wide array of materials, including, but not limited to, silicon, rubber, plastic or other polymer-based materials, or other suitable materials as is known in the art. The two tubing portions 102, 104 can be made of any suitable materials, as long as the hard tubing portion 104 of the cannula device 100 is very lightweight, resistant to kinking, and doesn't become twisted easily. Further, the soft tubing portion 102 of the cannula device 100 must be very flexible and soft to ensure the greatest performance and comfort for the patient/user 118. Accordingly, the cannula device 100 prevents bending or twisting of the hard tubing portion 104, however, the soft tubing portion 102 remains flexible and comfortable to prevent irritation during use. This is an improvement in comparison to conventional devices wherein the tubing can be twisted, bent, or become kinked, or causes irritation and is uncomfortable to wear.

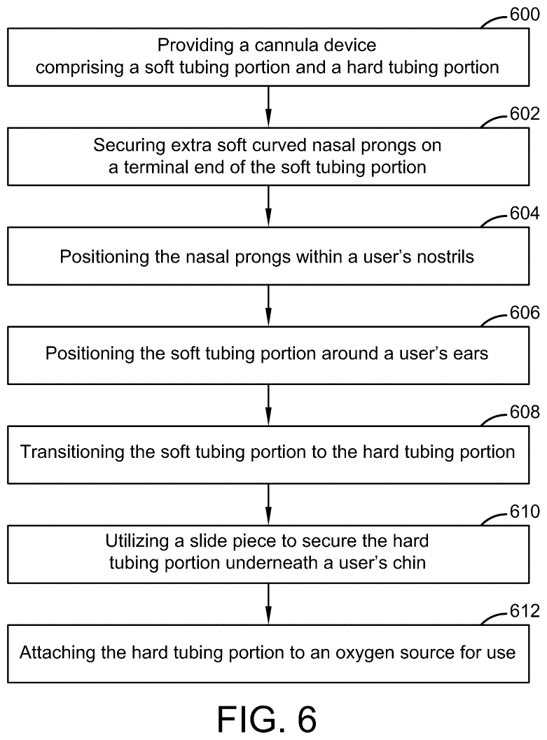

FIG. 6 illustrates a flowchart of the method of maintaining comfort without restricting airflow through a cannula. The method includes the steps of at 600, providing a cannula device comprising a soft tubing portion and a hard tubing portion. The method also comprises at 602, securing extra soft curved nasal prongs on a terminal end of the soft tubing portion. Further, the method comprises at 604, positioning the nasal prongs within a user's nostrils. The method also comprises at 606, positioning the soft tubing portion around a user's ears. The method also comprises at 608, transitioning the soft tubing portion to the hard tubing portion. The method comprises at 610, utilizing a slide piece to secure the hard tubing portion underneath a user's chin. Finally, the method comprises at 612, attaching the hard tubing portion to an oxygen source for use.

Certain terms are used throughout the following description and claims to refer to particular features or components. As one skilled in the art will appreciate, different users may refer to the same feature or component by different names. This document does not intend to distinguish between components or features that differ in name but not structure or function. As used herein “cannula device”, “nasal cannula device”, and “device” are interchangeable and refer to the cannula device 100 of the present invention.

Notwithstanding the forgoing, the cannula device 100 of the present invention can be of any suitable size and configuration as is known in the art without affecting the overall concept of the invention, provided that it accomplishes the above stated objectives. One of ordinary skill in the art will appreciate that the cannula device 100 as shown in FIGS. 1-6 is for illustrative purposes only, and that many other sizes and shapes of the cannula device 100 are well within the scope of the present disclosure. Although the dimensions of the cannula device 100 are important design parameters for user convenience, the cannula device 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A cannula device that provides a user with a modified nasal cannula, the cannula device comprising:
a soft tubing portion which is soft and flexible and provides comfort to a user's face; and
a hard tubing portion which is non-kinking tubing;
wherein the hard tubing portion is connected to the soft tubing portion; and
further wherein the soft tubing portion and the hard tubing portion attaches to an oxygen source and delivers a steady stream of medical-grade oxygen to a user's nose.

2. The cannula device of claim 1, wherein the cannula device is utilized with a portable oxygen system.

3. The cannula device of claim 2, wherein the cannula device comprises two parallel and hollow tubing portions for delivering oxygen to a user.

4. The cannula device of claim 3 further comprising a plurality of indicia.

5. The cannula device of claim 4, wherein the soft tubing portion encompasses a facial portion of the cannula device, which runs from a user's nose to around a user's ears and above a jawline.

6. The cannula device of claim 5, wherein the soft tubing portion is made of an extra soft, 2.10 mt medical grade PVC tubing.

7. The cannula device of claim 6, wherein the soft tubing portion comprises extra soft curved nasal prongs.

8. The cannula device of claim 7, wherein the extra soft curved nasal prongs are installed on a terminal end of the soft tubing portion.

9. The cannula device of claim 8, wherein the extra soft curved nasal prongs are molded from a very soft, pliable rubber, plastic or similar material (PVC) to prevent irritation to a user's nasal passages.

10. The cannula device of claim 9, wherein the extra soft curved nasal prongs are installed with a standard 4 mm internal diameter connector.

11. The cannula device of claim 10, wherein the extra soft curved nasal prongs are configured to sit just inside a user's nostrils.

12. The cannula device of claim 11, wherein the soft tubing portion is secured to a hard tubing portion via a connector.

13. The cannula device of claim 12, wherein the hard tubing portion is fabricated from a material more rigid than the soft tubing portion material is made from.

14. The cannula device of claim 13, wherein a slide piece is attached to the hard tubing portion to secure the hard tubing portion under a user's chin.

15. The cannula device of claim 14, wherein two hard tubing portions are then joined into one piece with a standard flared connection.

16. The cannula device of claim 15, wherein the cannula device delivers medical oxygen at a flow rate of 2-4 liters/min and delivers FiO2 of 0.28-0.36, respectively.

17. A cannula device that provides a user with a modified nasal cannula, the cannula device comprising:
a soft tubing portion which is soft and flexible and provides comfort to a user's face; and
a hard tubing portion which is non-kinking tubing;
wherein the soft tubing portion encompasses a facial portion of the cannula device, which runs from a user's nose to around a user's ears and above a jawline;
wherein the soft tubing portion comprises extra soft curved nasal prongs which are installed on a terminal end of the soft tubing portion;
wherein the soft tubing portion is secured to a hard tubing portion via a connector;
wherein the hard tubing portion is fabricated from a material more rigid than the soft tubing portion material is made from;
wherein a slide piece is attached to the hard tubing portion to secure the hard tubing portion under a user's chin;
wherein two hard tubing portions are then joined into one piece with a standard flared connection; and
further wherein once connected together through the standard flared connection, the hard tubing portion is then attached to an oxygen source to deliver a steady stream of medical-grade oxygen to a user's nose.

18. The cannula device of claim 17, wherein the soft tubing portion is made of an extra soft, 2.10 mt medical grade PVC tubing.

19. The cannula device of claim 17, wherein the extra soft curved nasal prongs are installed with a standard 4 mm internal diameter connector.

20. A method of maintaining comfort without restricting airflow through a cannula, the method comprising the following steps:
providing a cannula device comprising a soft tubing portion and a hard tubing portion;
securing extra soft curved nasal prongs on a terminal end of the soft tubing portion;
positioning the nasal prongs within a user's nostrils;
positioning the soft tubing portion around a user's ears;
transitioning the soft tubing portion to the hard tubing portion;
utilizing a slide piece to secure the hard tubing portion underneath a user's chin; and
attaching the hard tubing portion to an oxygen source for use.
